(19) United States
(12) Patent Application Publication
(10) Pub. No.: US 2021/0375478 A1
(43) Pub. Date: Dec. 2, 2021

(54) SYSTEM AND METHOD FOR UP-TO-DATE NUTRIENT DATABASE MANAGEMENT AND NUTRIENT ASSESSMENT

(71) Applicant: Nutrimedy, Brookline, MA (US)

(72) Inventors: Karolina Starczak, Beverly, MA (US); Christopher Hamling, Minneapolis, MN (US); Mallory P. Franklin, Minneapolis, MN (US)

(21) Appl. No.: 16/948,953

(22) Filed: Oct. 7, 2020

Related U.S. Application Data
(60) Provisional application No. 63/031,513, filed on May 28, 2020.
(60) Provisional application No. 63/031,516, filed on May 28, 2020.

(51) Int. Cl. G16H 50/70; G16H 70/20; G16H 70/60; G16H 50/20; G06Q 30/06; G16H 70/40; G16H 10/20; G16H 20/60; G06F 16/23; G06F 16/51; G06F 16/953; G06K 7/14; G06K 7/10; G06K 9/78; G01S 19/01

(52) U.S. Cl. CPC G16H 50/70; G06Q 50/26; G16H 70/60; G16H 50/20; G06Q 30/0631; G16H 70/40; G16H 10/20; G16H 20/60; G06F 16/2379; G06F 16/51; G06F 16/953; G06K 7/1413; G06K 7/10722; G06K 9/78; G01S 19/01; G16H 70/20

(57) ABSTRACT

A system and method for up-to-date nutrient database management and nutrient assessment with personalized feedback was created by applying artificial intelligence and machine learning algorithms. This system and method allow for the collection of food intake information, generation and storage of new food records with nutritional data and portions, aggregation of complete micronutrient and macronutrient data with real-time feedback to accurately and efficiently assess nutrient intake when compared to nutrient goals.

### RELATED APPLICATION

The present application claims the benefit of U.S. Provisional Application Nos. 63/031,516 and 63/031,513, filed May 28, 2020, which are hereby incorporated herein in their entireties by reference.

### TECHNICAL FIELD

The technology relates to the general field of healthcare and has certain specific applications to nutrient database management and nutrient assessment.

### BACKGROUND

Collecting accurate dietary intake is a major challenge. Current tools for assessing dietary intake include food frequency questionnaires, food diaries, food records, and diet recalls. These tools can be time consuming, require accurate recall of previous food intake, they can be limited to foods included in the questionnaire, and/or they require manually looking up foods from a nutrient database to collect nutrient composition data. Fully relying on self-reported portion sizes presents issues with accuracy. Another part of the challenge is maintaining an up-to-date nutrient database with relevant portion sizes since new products are continually added and some food products may be reformulated affecting nutrition data.

Adhering to dietary recommendations is a continual challenge for those managing a chronic condition or people who want to track dietary intake against specific goals to improve their health through nutrition. Nutrition recommendations or dietary restrictions can be complicated and challenging to follow. There is also no straightforward way to assess dietary intake goals against what a person consumes, which is further complicated by the growing number of food products. While nutrient databases such as the USDA database (“Food and Nutrition Information Center” at nal.usda.gov) can be comprehensive, they lack the consistency standards needed to provide accurate nutritional information. Many USDA entries (fdc_ids) are missing key nutrients or utilize a “100 g” default serving size, terminology that is unsuitable for many users.

Thus, there is a present need in the art for a means of capturing dietary intake through a consumer-friendly meal log with the capability to assess the food items, add new food items automatically to a database, accurately collect the nutrient information, or compare collected nutrient data against personalized dietary intake recommendation values will provide information needed to monitor nutrient intake.

### SUMMARY

The present disclosure comprises novel software-based services that provide a multifaceted approach to improve up to date nutrient database management and assessment of dietary data against personalized dietary intake recommendations. Features include meal image assessment, presentation of AI enabled portion suggestions, automated database expansion system (such as NuDB), and real-time assessment of intake against dietary recommendations.

Photos taken with a mobile device camera or uploaded on the web application will be used to identify food items either via AI capabilities for image recognition or by uploading the UPC-A barcode. Identified food item names will be displayed and nutrient information will be aggregated and displayed for the identified foods using the NuDB optimized from the existing food databases. Using a computer vision model, the meal log identifies food items via a mobile device camera, image upload, or UPC-A barcode to automatically retrieve nutritional information about a meal from the NuDB.

The meal log workflow may involve: 1) Create an interface to capture an image using the mobile device camera or upload an image or UPC-A barcode. 2) The computer vision model generates a list of food items that were identified in the image or UPC-A barcode. 3) Food items from the computer vision model are used to search the database and options that are matched from the database are displayed in a list. 4) The list of displayed food will be selectable and prompt the selection of suggested options for the serving size unit and enter the quantity. If no item is available in the database, the machine learning tool generates a record from other existing databases. 5) Nutrient information that corresponds to the food items captured in the image is aggregated, displayed, and compared to dietary intake recommendations with visual cues to display if the intake data is in range, above range, or below range based on personalized dietary intake recommendation values.

With the inconsistent and varied record types contained in the standard USDA database, the strategy for building a reliable set of serving sizes and labels varies depending on the input method of the record(s) found. In the ideal case when a “FNDDS Survey” or “SR Legacy” record is found, the portions are reliable and added to the NuDB for the given food item. For the majority of cases when a matching record of those types cannot be found, “Branded” records are searched with each one containing its own singular serving size as entered on a nutritional label. Since our goal is to offer a variety of usable choices, the top branded results and their respective serving sizes are queried, providing a more robust set of serving sizes for the NuDB. Once this set is created, the serving size results must be filtered for typos, plurals, duplicates, and other text inconsistencies and the sizes are scaled to a single unit of the given portion label. To add additional consistency and reliability, default portion sizes for “1 cup”, “1 oz”, and “1 tbsp” are added to all records provided that they do not already have a matching portion value.

Similar to portion database creation, when a match is found in “FNDDS Survey” or “SR Legacy” records of the USDA database, the nutrient information returned is reliable and used in the NuDB. In the cases where “Branded” results must be used, a set of the top matching records are accumulated, and the median value of each nutrient field is used as the true value in the NuDB. This is necessary due to the inconsistency of branded results in the USDA database, as they are entered via image upload of nutritional labels and not carefully curated, which can lead to missing fields and failed optical character recognition. In addition, the description field used to search branded records are commonly misleading, allowing for the possibility of incorrect nutritional fields that are handled by removing outliers and using median values.

Natural language processing algorithms automatically parse inconsistent text fields, merge and/or discard duplicate entries, and detect anomalies in the source database, such as the USDA database. Functioning as a cache for previously searched food items, the database contains fdc_ids that only include accurate nutritional and portion information as automatically selected by our algorithms. Due to the ever-expanding pool of food options, a machine learning tool automates a self-improvement feature to the database. When a food item matching the searched keywords cannot be found in the database, a text search algorithm is used to identify an accurate set of matching records from another database, such as the USDA database, and creates a new composite record that is added to the database. A natural language processing search algorithm identifies a set of results from the core USDA database in order to create a new nutritional record for the requested food item to be added to the NuDB. To maintain the integrity of the NuDB, each new record added by the machine learning tool needs to meet the standards for serving size and nutrient composition. To control for this, when a food item is not found in NuDB, algorithms generated above to create NuDB will be overlaid onto the food item search into the USDA database. Food items identified from the machine learning tool will be consistent with the standards generated for NuDB. Invalid records due to typos will be resolved by a text string matching algorithm.

Once the nutritional information is collected from either an existing or newly created record and the corresponding portion size, the sum of each micronutrient and macronutrient per meal and per day is compared to the individual personalized dietary intake recommendation values. The system then visually represents this comparison by displaying the percentage of the individual personalized dietary intake recommendation values that can be entered through the food log and color coding the visual representation of the percentages. When no value or percent has been consumed on that day, the display color is grey. When the percent consumed is less than 75% the display color is yellow. When the percent consumed is over 75% and under 105%, the display color is green. When the percent consumed is over 105%, the display color is red. The visual displays can also show a weekly and monthly view to visually represent the user's adherence to the personalized dietary intake recommendation values. The user can also click on each color to display a list of food items that contributed to the macronutrient or micronutrient adherence by day, week, month, or for a specific date range. These lists are displayed by the specific macronutrient or micronutrient value in descending order.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is an illustration of food log workflow.

FIG. 2 is an illustration of a NuDB database.

FIG. 3 is an illustration of a micronutrient and macronutrient feedback.

FIG. 4 is an illustration of a new record in NuDB along with portion size

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

By creating a way to collect user food intake information via multiple collection methods (mobile device camera upload, image upload on the web application, UPC-A barcode, or entering by typing the food item into a text field), the present disclosure allows for needed flexibility to accommodate multiple situations and removes barriers for food log entries. Whether a user is on their mobile device, tablet, or computer, they can enter the food item into the food log quickly and easily. It also allows the user to add a food item regardless of the food item being in a specific dish, in a box, or not having access to a camera to capture the image.

NuDB is used throughout to refer to a nutrition database maintained by the device, application, system or other implementation of the present invention. Though some embodiments of the present disclosure may use a “NuDB” database model (append only, key/value store), it should be understood that a wide variety of database models may be used to implement the NuDB (nutrition database) discussed herein.

When the user enters a meal log entry, the NuDB allows for real-time identification and aggregation of macronutrient and macronutrient data. This removes the need for the user to parse through a list of food items that would normally come from a text search result. It also creates a more complete record since many search results from other databases, such as the USDA database, produce incomplete records. If the user has a restriction or a dietary modification that is specific to a micronutrient(s), retrieval of food items with incomplete records would result in inaccurate aggregated nutrient totals. This would result in the user making dietary decisions based on inaccurate information, which can have adverse health effects. If the food log record is being used by the healthcare team to measure compliance with dietary modifications, it is crucial that the micronutrient and macronutrient values be accurate. As NuDB grows due to the creation of new composite records that are added to the database, it allows the system to learn additional information about food relation decision making as it pertains to existing medical conditions and personalized dietary intake recommendations. For example, if users with Congestive Heart Failure who have a sodium restriction in place tend to over consume sodium the most when eating meals that contain fried items, that insight can be applied to proactive feedback and coaching for the user. Identification of these higher risk situations or food items can provide personalized and population-based lists that can potentially influence how we provide diet-related education and even provide warnings to healthcare teams if the user starts to repeatedly add food items from these higher risk foods.

Due to the real-time automated nature the food log, NuDB, and feedback system can be used in real-world decision-making capacities. When users go to restaurants, events, outings, or the supermarket, they can easily create a food entry and see how the food compares to their personalized dietary intake recommendations. For people with specific dietary needs, making decisions is difficult and this system can provide support in daily dietary informed decision making. NuDB's capabilities can also be connected to mobile GPS functionality to provide location specific recommendations based on the personalized dietary intake recommendations. If the user chooses to activate location tracking, the system can generate alerts if they are within a specific radius of a food establishment or market that has food items appropriate for the user. In embodiments, the system may be configured to generate alerts for other (non location based) criteria, such as time from last entry or others. For example, these automated messages may be set to only become active if the user has not entered a food item into the food log within the previous 3 hours. By providing another option for the user other than to parse through menus, the system may remove another barrier to dietary compliance.

Since the system contains the micronutrient and macronutrient data that is used to compare actual intake to personalized dietary intake recommendations, it can also be used to suggest modifications. If the user consistently enters food items that are outside of the parameters created by the personalized dietary intake recommendations, the system can be applied to find potential substitutions if available. It is not uncommon for one brand of a food item to be higher in sodium or have more sugar than another. Once the system identifies a substitute it can prompt the user to suggest the modification. These substitution prompts can remove another barrier to compliance and save the user time in looking for potential substitutions.

The system may also allow the user to enter data points such as energy levels, medication, bowel movements, mood, food allergies and any other information they would like to track. Tracked information can also be connected to third party devices such as activity trackers, glucometers, and scales. The system can then not only identify food-drug interactions in real time and alert the user, but it can also provide insight into which food items result in the most positive feelings of wellness. While compliance to personalized dietary intake recommendations is important, certain foods may result in negative health effects that are difficult to link to diet. Having a system that can provide insight into which food items put the user at risk for food-drug interactions, allergic reactions, an increase in negative health effects such as bloating, fatigue, headaches, insomnia, constipation, joint pain, negative moods and many more can provide the user with information that allows them to have more control over their health. This information may be represented numerically or visually, such as with graphs or other illustrative graphics, to allow the user to see what they consumed on days when they reported feeling their best based on their inputs. Food items may also be organized into subcategories, either automatically or manually. Particular organization may be a default or adjusted according to the user's needs. In one example, it may provide two lists, one of all food items that frequently appear in the food log when the person is not feeling their best and a second list of all food items that frequently appear in the food log when the person is reporting positive data inputs such as having high energy.

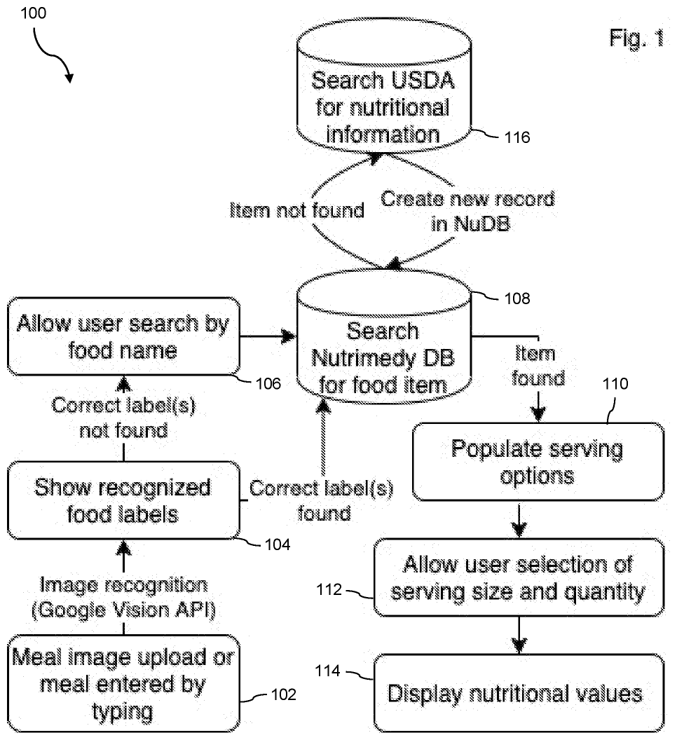

Referring now to FIG. 1, an illustration of food log workflow 100 is shown. A meal image or images of UPC code(s) associated with the meal or typed description may be entered at 102, as by a user or automatically populated by the system or by an external system. Food items are identified and displayed at 104. The system may adjust the image recognition used depending on the entry format. The user may correct the system at 106 if any of the displayed foods were incorrectly identified. Correct and correct food items are submitted to the nutrition database (NuDB) at 108. Items that are found are populated to the user's display as serving options as 110. The user is permitted to select the appropriate serving size and quantity at 112. Nutritional values associated with the entry are calculated and displayed at 114.

If an item is not found in the NuDB, external databases, such as the USDA nutritional information database, are searched for appropriate entries, at 116. Once an entry for the item is found, a new record is created in the NuDB at 108.

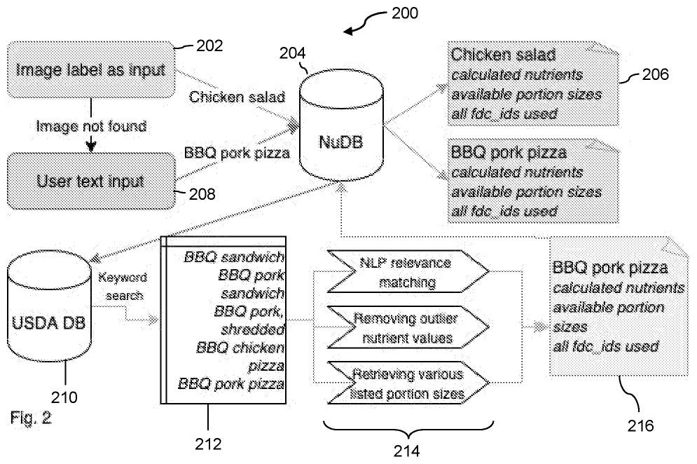

Referring now to FIG. 2, an illustration of a NuDB database executing an example workflow 200 is shown. In one example, an image 202 is identified by the NuDB 204 as chicken salad. In response, NuDB 204 generates a display of nutrition data 206 associated with the image provided. In another example, a text input 208 is received by NuDB 204. The text input 208 for “BBQ pork pizza” is not recognized by the NuDB and an external database 210, the USDA database in this example, is searched. In this example, a keyword search 212 is used, appropriate to the text entry 208 of the unknown food, but other search methods are envisioned as appropriate for the unknown entry and the target database. A number of quality measures 214 are used to ensure the new entry 216 to be generated for the NuDB 204 is adequate to the systems needs.

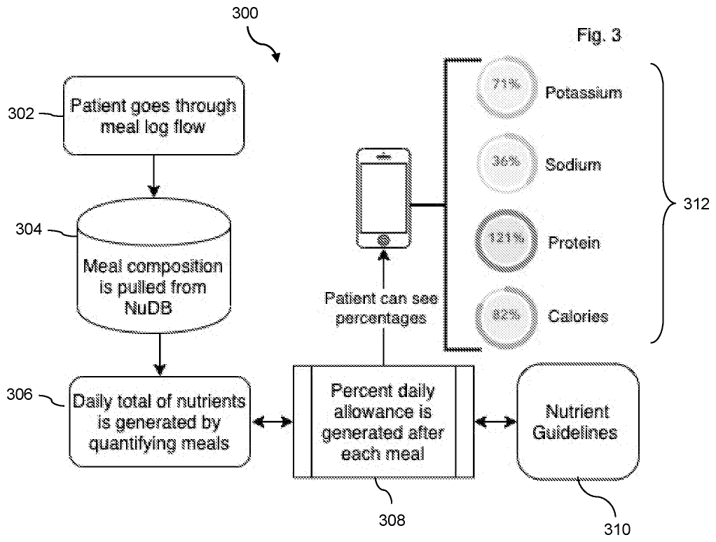

Referring now to FIG. 3, an example workflow 300 leading to a micronutrient and macronutrient feedback is shown. As the user enters their meal logs throughout the day 302, the NuDB calculates meal composition 304 and the system aggregates the totals throughout the day 306. Many users may find a day to be a convenient unit for tracking their totals according to guidelines (which are often provided as Daily Values) but other units may be used in embodiments. Periodically throughout the day, the system will generate a display 308 to illustrate for the user the progress toward and compliance with dietary recommendations 310. In this example, the display 308 is generated after each meal entry but other triggers or periodicities may be used in embodiments. The display may be graphically 312 presented to assist the user in quickly grasping where they are doing well and where they should make more careful choices. The graphics 312 may be a simple number and text display or, as in this example, colors, shapes, graphs, etc. may be used.

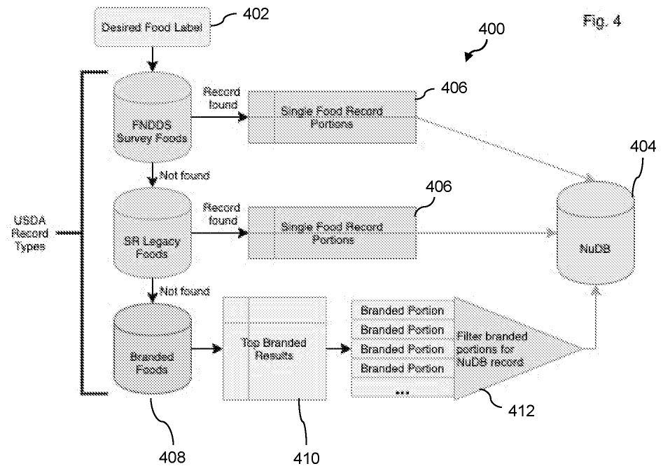

Referring now to FIG. 4, an illustration 400 of a new record generation in NuDB along with portion size is shown. A new food 402 is received by the system and the system begins a search of external databases to populate a new entry in the NuDB 404. Some standardized record types, such as FNDDS Survey records and SR Legacy records allow for a single record entry 406, as the record is already in a format the NuDB 404 can reliably employ. However, many foods only have branded records 408 available, which cannot be relied upon by NuDB 404. To generate an appropriate new record, the system collects the top branded results 410 for the target food 402 and then filters out 412 errors and duplicates. Information is standardized to a single serving and mean values for nutritional information.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A method for populating a nutrition database, comprising:
receiving an image;
identifying one or more food items in the image using a computer vision model;
retrieving nutrition data for each of the identified one or more food items from a nutrition database;
aggregating the nutrition data for all of the one or more food items in the image;
comparing the aggravated nutrition data with a dietary intake recommendation for a user;
generating a visual cue of the comparison between the aggregated nutrition data and the dietary intake recommendation; and
displaying names of the one or more food items, the aggregated nutrition data, and the generated visual cue of the comparison.

2. The method of claim 1, wherein receiving the image comprises uploading the image to a web application associated with the nutrition database.

3. The method of claim 1, further comprising taking the image with an associated image capture device.

4. The method of claim 1, wherein the image is at least one of a photograph or a barcode.

5. The method of claim 1, wherein the image is of a meal.

6. The method of claim 1, further comprising:
determining that at least one food item of the one or more food items is not in the nutrition database;
generating a new entry in the nutrition database for the at least one food item; and
populating the new entry by retrieving nutrition data related to the at least one food item from an external database.

7. The method of claim 1, wherein the aggregated nutrition data further comprises data from other food item entries made earlier in an entry period.

8. The method of claim 7, wherein the entry period is a day.

9. A method for optimizing nutrition database, comprising:
generating a new entry for a food item in the nutrition database;
retrieving a set of nutrition data associated with the food item from an external database;
determining an input method of the set of nutrition data retrieved from the external database is not “FNDDS Survey” and is not “SR Legacy”;
querying one or more branded results and one or more branded results serving sizes;
filtering the one or more branded results and the one or more branded results servings sizes;
scaling a serving size for the new entry to a single unit of a given portion label;
finding a median value for each nutrient value of the one or more branded results; and
using a median value of each nutrient value for the new entry.

10. A system for optimizing nutrition data, comprising:
a nutrition database that is optimized by:
generating a new entry for a food item in the nutrition database;
retrieving a set of nutrition data associated with the food item from an external database;
determining an input method of the set of nutrition data retrieved from the external database is not “FNDDS Survey” and is not “SR Legacy”;
querying one or more branded results and one or more branded results serving sizes;
filtering the one or more branded results and the one or more branded results servings sizes;
scaling a serving size for the new entry to a single unit of a given portion label;
finding a median value for each nutrient value of the one or more branded results; and
using a median value of each nutrient value for the new entry.

11. The system of claim 10, wherein the nutrition database is associated with a GPS.

12. The system of claim 10, wherein the system is configured to generate automated alerts.

13. The system of claim 12, wherein an automated alert is generated if a time without entry exceeds a threshold value.

14. The system of claim 10, wherein multiple branded entries are organized under one food item entry, and the system is configured to generate recommendations for brands according to suitability of a brand nutrition profile and a user intake recommendation.

15. The system of claim 12, wherein the system generates an automated alert for a possible food-drug interaction.

16. The system of claim 10, wherein the system receives data indicating a feeling of well-being of a user and correlates the received data with data indicating a diet of the user and provides an indication to the user of foods with associated changes in data indicating the feeling of well-being of the user.
